(19) United States
(12) Patent Application Publication
(10) Pub. No.: US 2025/0212839 A1
(43) Pub. Date: Jul. 3, 2025

(54) KIT FOR ASSEMBLING A PIECE OF FURNITURE

(71) Applicant: MEOW MEWS INC., Allentown, PA (US)

(72) Inventor: Netta Dor SHALGI, Allentown, PA (US)

(21) Appl. No.: 18/853,009

(22) PCT Filed: Apr. 4, 2023
(86) PCT No.: PCT/IL2023/050368

§ 371 Date: Sep. 30, 2024

Related U.S. Application Data
(60) Provisional application No. 63/327,395, filed on Apr. 5, 2022.

(51) Int. Cl. A01K 1/03; A01K 1/01; A01K 15/02; A01K 29/00; A61B 5/16; F16B 12/40

(52) U.S. Cl. CPC A01K 1/033; A01K 1/0107; A01K 29/005; F16B 12/40; A01K 15/024; A61B 5/165; A61B 2503/40; F16B 2012/403

(57) ABSTRACT

A ready made furniture or kit for assembling a piece of furniture optionally with computer-implemented devices for monitoring behavior of at least one habitant, which may be feline; said system comprising: a plurality of furniture connectors assemblable into mountable furniture assembly units; at least one linking member configured for interconnecting said vertically mountable assembly units and sensors and data processing for monitoring behaviour.

### FIELD OF THE INVENTION

The present invention generally pertains to modular furniture and, more particularly, modular furniture creating a living environment for one or more cats.

### BACKGROUND OF THE INVENTION

Environmental needs include a cat's physical surroundings (indoors, outdoors, or both) and social interactions with humans and other pets. Cats often do not express obvious signs of stress, pain, or sickness that we can easily recognize. If the pet's owner is proactive and meets appropriate environmental needs throughout a cat's life, he or she can potentially avoid environmental stressors that can cause unwanted behaviors and even impact medical health.

Nowadays, the needs of cats have changed little from those of their wild ancestor, Felis lybica, the African wildcat. Specifically, cats are solitary hunters, spending much of their day searching the environment for hunting opportunities. They need to protect themselves from perceived dangers, which include unfamiliar individuals or environment. Cats are territorial animals. They feel threatened when their territory is disturbed, either by another animal or physically. Cats use scent, posturing, and vocalizations to communicate their unhappiness if they feel threatened. Cats have a superior sense of smell and hearing. Stress can occur due to strong or strange smells or sounds, which are undetectable or insignificant to us. Cats are social animals, but their social structure differs from ours. Cats may be content as a single cat or living with other cats, preferably related cats such as siblings.

The environment provided for cats, especially in a typical apartment or a house, is limited and very different from cats' natural outdoors and trees habitat. In order to provide an enriching experience which is closer to nature and is more intriguing and challenging for the cat or cats, cat owners typically invest in cat furniture, such as cat condos, scratching posts, water fountains and toys. The cat's furniture then becomes part of the household.

However, typical cat furniture and accessories lack modern design and esthetics. Often, they are not robust enough and do not last long; or, for long-term use, have very limited or no adjustability, have limited functionality, and are lacking in dynamic capabilities.

According to recommendations of veterinarians (see, for example, Sarah L H Ellis e al, AAFP and ISFM Feline Environmental Needs Guidelines, Journal of Feline Medicine and Surgery (2013) 15, 219-230), for a cat, a safe place is a private and secure area, often in a raised location. These features give the cat a sense of enclosure, isolation or seclusion. A safe place is one that a cat can retreat to so that it feels protected. If the cat cannot see a potential threat, it feels safer, even if its whole body is not fully concealed. When a cat is relaxed, a safe place can also function as a resting or sleeping area.

U.S. Pat. No. 5,806,464 discloses a modular pet furniture comprising one or more vertical columns constructed from a series of interconnected column segments supporting one or more platforms upon which a pet may rest. Tubular column segments are interconnected within mating sockets provided on the platforms as well as on lateral connectors used for interconnecting and mutually supporting adjacent modular columns. Each platform as well as the entire column upon which each platform is supported may be rotated to any desired angle with respect to an adjacent support column to vary the size and configuration of the assembly.

Therefore, there is a long-felt need to provide vertically arranged pieces of cat furniture meeting requirements of cat natural behavior.

### SUMMARY OF THE INVENTION

It is hence one object of the invention to disclose a furniture connector comprising: (a) first and second articles having at least one first terminal face each; said terminal portion has a blind bore thereon; (b) an intermediate member defined by contralaterally arranged first and second engaging faces; said intermediate member having a first through bore getting through said first and second engaging faces; (c) a dowel insertable into said blind bores of said first and second articles and said first through bore.

It is hence one object of the invention to disclose the terminal face of terminal first article engageable to first face of said intermediate member and second engaging face of said intermediate member is engageable to said terminal face of said second article by means of a plug-socket connectors such that said blind bores of said first and second articles and said first through bore of said intermediate member are coaxially arranged and configured for receiving said dowel in a releasable manner.

Another object of the invention is to disclose at least one of said first and second articles has a second through bore transversely oriented to said blind bore; said dowel has a third through bore at least one terminal of said dowel cooperatively disposed such that, when said dowel is inserted into said blind bore of at least one of said first and second articles, said second and third through bore are arranged coaxially and a stud releasably securable therewithin is insertable.

A further object of the invention is to disclose the third through bore having a middle bore portion and terminal portions adjacent thereto; said terminal portions are tapered such that said stud is frictionally securable in said middle portion of said third trough bore.

A further object of the invention is to disclose at least one of said plug-socket connectors comprising a pair of surfaces facing each other; at least one of said surfaces has at least one pin projecting therefrom and releasably insertable into at least one hole on a facing surface.

A further object of the invention is to disclose at least one of said first and second articles having an axisymmetrical body.

A further object of the invention is to disclose the axisymmetrical body having a middle portion and terminal portions adjacent thereto. The axisymmetrical body gradually grows in diameter thereof from said middle portion to said terminal portion.

A further object of the invention is to disclose a side surface of said axisymmetrical body which is coiled with a rope.

A further object of the invention is to disclose a rope fastener that holds the rope end with a ring and a screw that penetrates the rope.

A further object of the invention is to disclose the said axisymmetrical body having at least one fourth through bore having a middle bore portion and terminal portions adjacent thereto; said terminal portions are tapered. At least one terminal of said rope is inserted into said at least one forth through bore such that said rope is frictionally securable in said middle portion of said forth though bore.

A further object of the invention is to disclose the dowel threadly insertable into said blind bores of said first and second articles and said first through bore.

A further object of the invention is to disclose at least one of said plug-socket connectors which is threaded.

A further object of the invention is to disclose at least one of first and second articles having a second terminal face placeable on or securable to a surface of interest.

A further object of the invention is to disclose the surface of interest selected from the group consisting of a floor, a wall, a ceiling and any combination.

A further object of the invention is to disclose a kit for assembling a piece of furniture or at least a part thereof. The foresaid kit comprises: (a) a plurality of furniture connectors assemblable into vertically mountable furniture assembly units; each furniture connector of said assembly unit further comprising: (i) first and second articles having at least one terminal face each; said terminal portion has a blind bore thereon; (ii) an intermediate member defined by contralaterally arranged first and second engaging faces; said intermediate member having a first through bore getting through said first and second engaging faces; (iii) a dowel insertable into said blind bores of said first and second articles and said first through bores; said terminal face of terminal first article is engageable to first face of said intermediate member and second engaging face of said intermediate member is engageable to said terminal face of said second article by means of a plug-socket connectors such that said blind bores of said first and second articles and said first through bore of said intermediate member are coaxially arranged and configured for receiving said dowel in a releasable manner; and (b) at least one linking member configured for interconnecting said vertically mountable assembly units.

A further object of the invention is to disclose a kit for assembling a piece of furniture or at least a part thereof. The aforesaid kit comprises: (a) a plurality of furniture connectors assemblable into mountable furniture assembly units; each furniture connector of said assembly unit further comprising: (i) first and second articles having at least one terminal face each; said terminal portion has a blind bore thereon; (ii) an intermediate member defined by contralaterally arranged first and second engaging faces; said intermediate member having a first through bore getting through said first and second engaging faces; (iii) a dowel insertable into said blind bores of said first and second articles and said first through bores; said terminal face of terminal first article is engageable to first face of said intermediate member and second engaging face of said intermediate member is engageable to said terminal face of said second article by means of a plug-socket connectors such that said blind bores of said first and second articles and said first through bore of said intermediate member are coaxially arranged and configured for receiving said dowel in a releasable manner; at least one of said first and second articles has a second through bore transversely oriented to said blind bore; said dowel has a third through bore at at least one terminal of said dowel cooperatively disposed such that, when said dowel is inserted into said blind bore of at least one of said first and second articles, said second and third through bore are arranged coaxially and a stud releasably securable therewithin is insertable; and (b) at least one linking member configured for interconnecting said vertically mountable assembly units.

A further object of the invention is to disclose a computer-implemented device for monitoring behavior of at least one habitant. The aforesaid system comprises: (a) a sensor arrangement further comprising a sensor selected from the group consisting of a touch pad, a microphone, a camera, a motion sensor, a temperature sensor, a blood oxygen sensor, a laser rangefinder, a CO₂ sensor, an RFID antenna, a gyroscopic sensor, a pressure sensor, an illumination sensor, a heart-beat rate sensor and any combination thereof; (b) a processor; (c) at least one user unit further comprising a user interface; (d) a memory unit/server storing instructions to said processor to perform: (i) sensing data relating to behavior of at least one habitant; (ii) accumulating said data within said memory unit; (iii) recognizing a typical behavior pattern of said habitant; (iv) correlating currently sensed data with said typical behavior pattern; (v) sending a report relating to abnormal behavior of said habitant to said at least one user device.

Performing said instructions of recognizing a typical behavior pattern of said habitant and correlating currently sensed data with said typical behavior pattern comprises applying a convolutional neural network algorithm to said accumulated data.

A further object of the invention is to disclose the sensor arrangement, processor, at least one user unit and memory unit which are interconnected with each other by means of a communication protocol selected from the group consisting of: a wire connection, BlueTooth, Wi-Fi, broad band internet, an optical fiber and any combination thereof.

A further object of the invention is to disclose a computer-implemented method of monitoring behavior of at least habitant. The aforesaid method comprises steps of: (a) providing a device for monitoring behavior of at least one habitant further comprising: (i) a sensor arrangement further comprising a sensor selected from the group consisting of a touch pad, a microphone, a camera, a motion sensor, a temperature sensor, an blood oxygen sensor, laser distance meters, a CO₂ sensor, an RFID antenna, a scale, a gyroscopic sensor, a pressure sensor, a illumination sensor, a heart-beat rate sensor and any combination thereof; (ii) a processor; (iii) at least one user unit further comprising a user interface; (iv) a memory unit storing instructions to said processor; (b) sensing data relating to behavior of at least one habitant; (c) accumulating said data within said memory unit; (d) recognizing a typical behavior pattern of said at least one habitant; (e) correlating currently sensed data with said typical behavior pattern; (f) sending a report relating to abnormal behavior of said at least habitant to said at least one user device; performing said instructions of recognizing a typical behavior pattern of said at least one habitant and correlating currently sensed data with said typical behavior pattern comprises applying a convolutional neural network algorithm to said accumulated data.

A further object of the invention is to disclose a cloud-based computer-implemented system for monitoring habitant behavior. The aforesaid system comprises: (a) a plurality of devices for monitoring behavior of at least habitant; each said system further comprising (i) a sensor arrangement further comprising a sensor selected from the group consisting of touch pad, a microphone, a camera, a motion sensor, a temperature sensor, an blood oxygen sensor, laser ranfinder, a CO₂ sensor, an RFID antenna, a scale, a gyroscopic sensor, a pressure sensor, an illumination sensor, a heart-beat rate sensor and any combination thereof; (ii) a processor; (iii) at least one user unit further comprising a user interface; (iv) a memory unit storing instructions to said processor to perform: (1) sensing data relating to behavior of at least habitant; (2) accumulating said data within said memory unit; (b) cloud-based software means configured for: (i) interrogating said data accumulated in said memory units of said plurality of said systems for monitoring behavior of habitant; (ii) recognizing a typical habitant pattern; (iii) correlating currently sensed data with said typical behavior pattern; (iv) sending a report relating to abnormal behavior of said habitant to said at least one user device belonging to said at least one user device belonging to a corresponding system for monitoring behavior of habitant; performing said instructions of recognizing a typical behavior pattern and correlating currently sensed data with said typical behavior pattern comprises applying a convolutional neural network algorithm to said accumulated data.

A further object of the invention is to disclose the sensor arrangement, processor, at least one user unit, memory unit and cloud-based software means which are interconnected with each other by means of a communication protocol selected from the group consisting of: a wire connection, BlueTooth, Wi-Fi, broad band internet and any combination thereof.

A further object of the invention is to disclose a method of monitoring habitant behavior. The aforesaid method comprises steps of: (a) providing a cloud-based computer-implemented system for monitoring habitant behavior; said system comprising: (i) a plurality of devices for monitoring behavior of habitant; each said system further comprising a sensor arrangement further comprising a sensor selected from the group consisting of touch pad, a microphone, a camera, a motion sensor, a temperature sensor, a blood oxygen sensor, a laser rangefinder, a CO₂ sensor, an RFID antenna, a scale, a gyroscopic sensor, a pressure sensor, an illumination sensor, a heart-beat rate sensor and any combination; (ii) a processor; (iii) at least one user unit further comprising a user interface; (iv) a memory unit; (b) cloud-based software means configured for: (i) interrogating said data accumulated in said memory units of said plurality of said systems for monitoring behavior of habitant; (ii) recognizing a typical behavior pattern; (iii) correlating currently sensed data with said typical behavior pattern; (iv) sending a report relating to abnormal behavior of said habitant to said at least one user device belonging to said at least one user device belonging to a corresponding system for monitoring behavior habitant; (b) sensing data relating to behavior of habitant by each of said plurality of said devices; (c) accumulating said data within said memory unit; (d) interrogating said data accumulated in said memory units of said plurality of said systems for monitoring behavior of habitant; (e) recognizing a typical habitant behavior pattern; (f) correlating currently sensed data with said typical behavior pattern; (g) sending a report relating to abnormal behavior of said habitant to said at least one user device belonging to said at least one user device belonging to a corresponding system for monitoring behavior of habitant; performing said instructions of recognizing a typical behavior pattern and correlating currently sensed data with said typical behavior pattern comprises applying a convolutional neural network algorithm to said accumulated data.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIGS. 1a to 1c are schematic cross-sectional views of a furniture connector (FIG. 1a), an article (FIG. 1b) and an intermediate member (FIG. 1c);

FIGS. 2a, 2b and 2c are schematic external, cross-sectional and exploded views of a furniture connector with a transversal securing studs;

FIGS. 3a and 3b are schematic cross-sectional and exploded views of a furniture connector with securing pins;

FIG. 3c is an isometric view of an intermediate member;

FIGS. 4a and 4b are schematic isometric and cross-sectional views of a dowel;

FIGS. 5a, 5b and 5c are schematic cross-sectional views of a furniture connector (FIG. 5a), an article (FIG. 5b) and an intermediate member (FIG. 5c) threadly connected to each other;

FIG. 6 is a schematic cross-sectional of a furniture connector provided with a threaded dowel therewithin;

FIGS. 7a to 7e are exemplary embodiments of articles 3a/3b shown in FIG. 1a;

FIGS. 8 to 16 present alternative embodiments of an assembled piece of furniture;

FIGS. 17a and 17b present assembled and exploded views of an end article provided with a sensor arrangement;

FIGS. 18a and 18b present assembled and exploded views of an intermittent member provided with a sensor arrangement; and

FIGS. 19 and 20 are flowcharts of alternative computer-implemented methods of monitoring habitant behavior.

FIGS. 21 and 22 present embodiments of the rope securing mechanism whereby a rope fastener is provided that holds the rope end with a ring and a screw that penetrates the rope.

### DETAILED DESCRIPTION OF THE INVENTION

The following description is provided, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide a furniture connector and a kit for assembling a piece of furniture.

The term “habitant” hereinafter refers to any climbing animal.

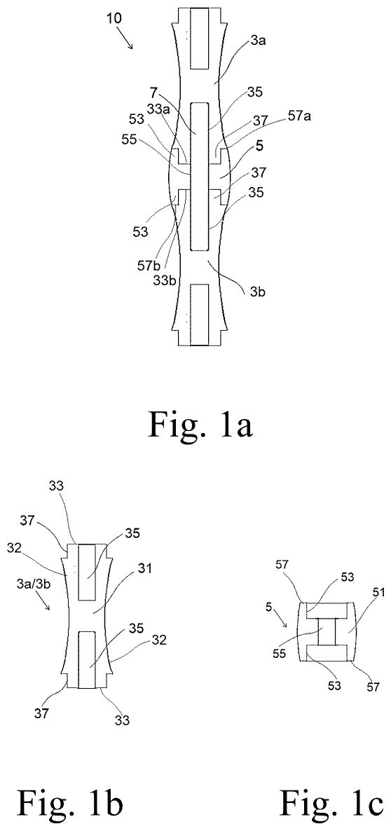

Reference is now made to FIGS. 1a to 1c presenting schematic cross-sectional views of a furniture connector (FIG. 1a), an article (FIG. 1b) and an intermediate member (FIG. 1c). Specifically, referring to FIG. 1, furniture connector 10 comprises articles 3a and 3b and intermediate member 5 placed therebetween. Numeral 7 refers to an internally disposed dowel. Article 3a/3b has middle portion 31 and at least one (two in FIG. 1b) terminal portion 32. Terminal face 33 is provided with blind bore 35. FIG. 1c shows intermediate member 5 having engaging faces 57 and through bore 55. Numeral 53 refers to female portion of the plug-socket connector configured for receiving male potion 37 (FIG. 1b).

Face 33a of first terminal article 3a is engageable to first face 57a of intermediate member 5. Second engaging face 57b of intermediate member 5 is engageable to terminal face 33b of second article 3b. Male portions 37 belonging to first and second articles 3a and 3b are inserted into female portions 53 of intermediate member 3 and form a plug-socket connection. Blind bores 35 of first and second articles 3a and 3b and first through bore 55 of intermediate member 5 are coaxially arranged and configured for receiving dowel 7 in a releasable manner.

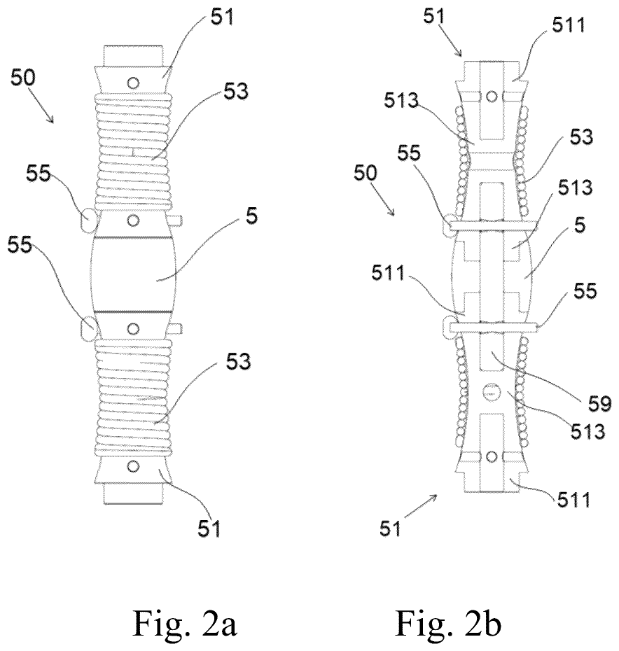

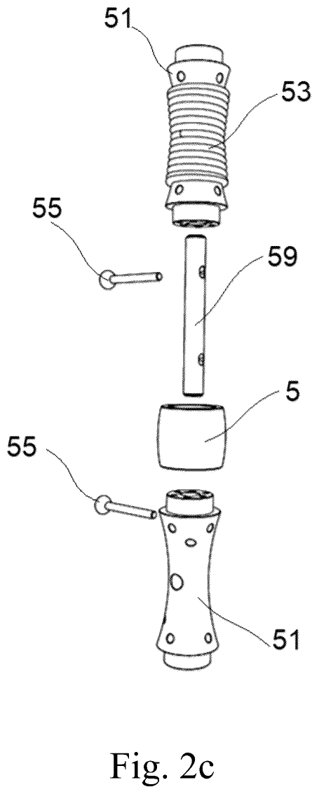

According to one embodiment of the present invention, the articles have axisymmetrical body 51 as shown, for example, in FIG. 2b. Axisymmetrical body 51 has a middle portion 513 and terminal portions 511 adjacent thereto. Axisymmetrical body 51 gradually grows in its diameter from middle portion 513 to terminal portions 511. The diameter profile of axisymmetrical body 51 provides self-compression of rope coil 53 in a natural manner.

Reference is now made to FIGS. 2a to 2c presenting an alternative embodiment of the present invention of a furniture connector which is provided with transversally oriented securing studs 55. FIGS. 2a, 2b and 2c are schematic external, cross-sectional and exploded views of a furniture connector, respectively. As shown in FIGS. 2b and 2c, dowel 59 has transversal bores configured receiving studs 55 which secure dowel 59 within articles 51. According to one embodiment of the present invention, articles 51 are coiled with ropes 53.

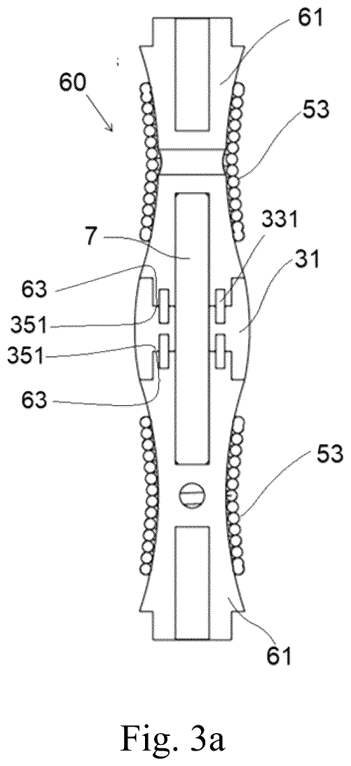

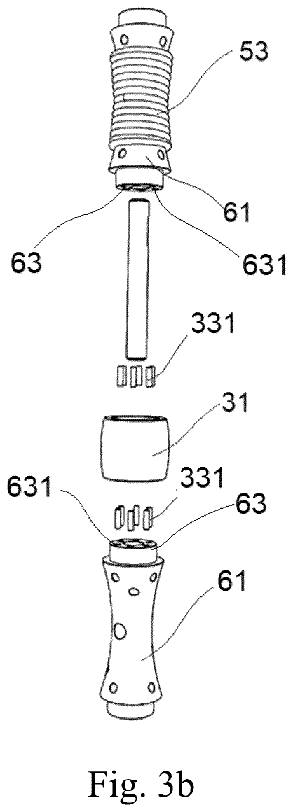

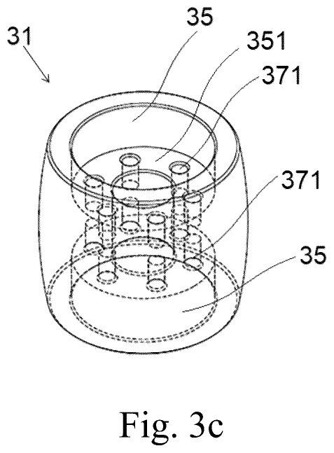

Reference is now made to FIGS. 3a to 3c presenting an alternative embodiment of the present invention of a furniture connector which is provided with longitudinally oriented pins 331. Faces 63 of articles 61 are provided with bores 631 while face 351 of intermediate member 31 with bores 371. Bores 371 and 631 are configured for receiving pins 331 in the process of assembling the connector. Combination of the embodiments shown in FIGS. 2a-2c and FIGS. 3a-3c is also in the scope of the present invention.

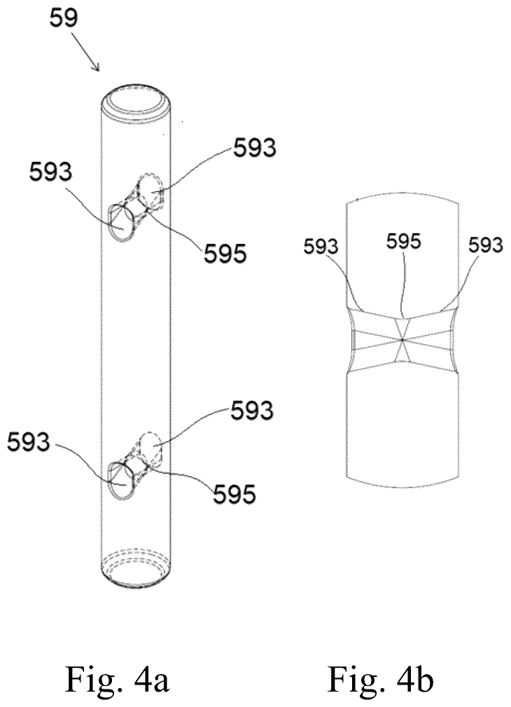

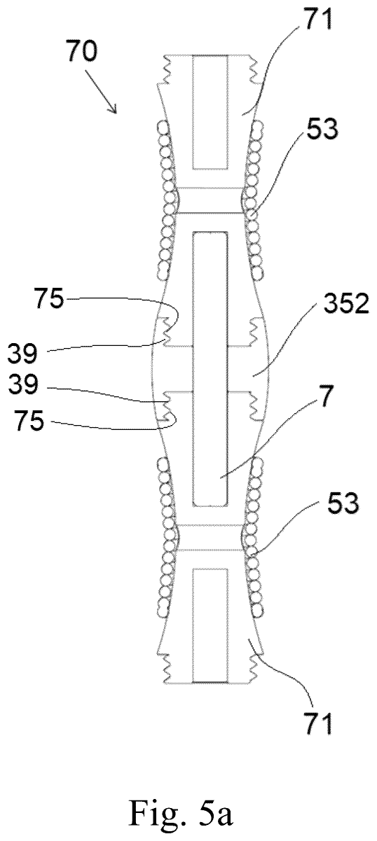

Reference is now made to FIGS. 4a to 4b presenting schematic isometric and cross-sectional views of dowel 59. The aforesaid dowel is provided with at least one trough bore. The bores have middle portion 595 and terminal portions 593. The geometry of the bores such that terminal portions 593 are tapered such that stud 55 (FIGS. 2a-2c) is frictionally securable in middle portion 595 of the through bore.

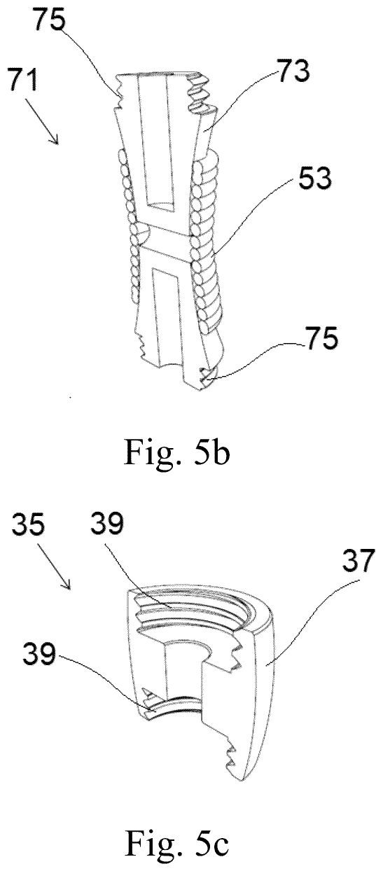

Reference is now made to FIGS. 5a to 5c presenting threadly securable furniture connector 70. Articles 71 are threadly are securable to intermediate member 35.

Specifically, male thread 75 is screwed into female thread 39. Similar to the previous embodiments, dowel 7 interconnects blind bores of within articles 71 via through bore within intermediate member 352.

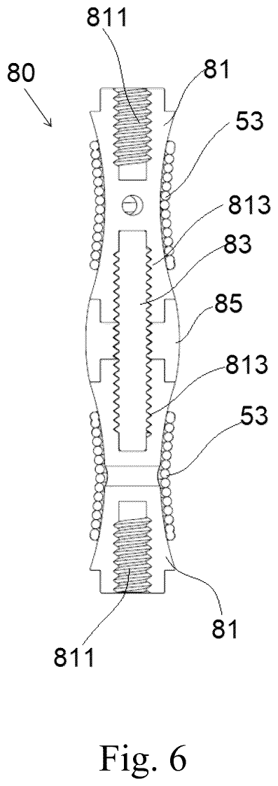

Reference is now made to FIG. 6 presenting threadly securable furniture connector 80. Articles 81 are provided with one (813) or two (811 and 813) threaded bores. Threaded dowel 813 when screwed into threaded bores 813 interconnects articles 81 and intermediate member disposed

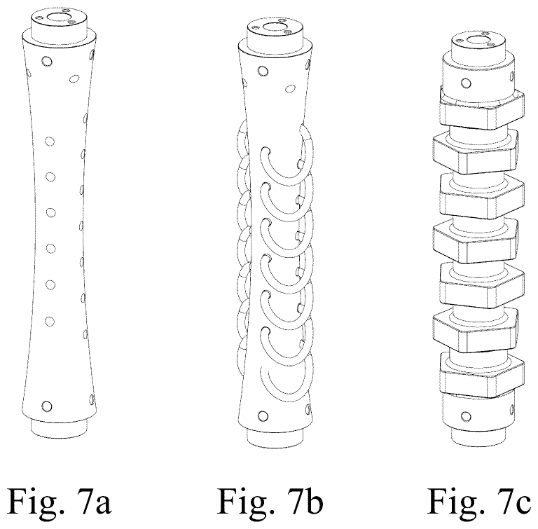

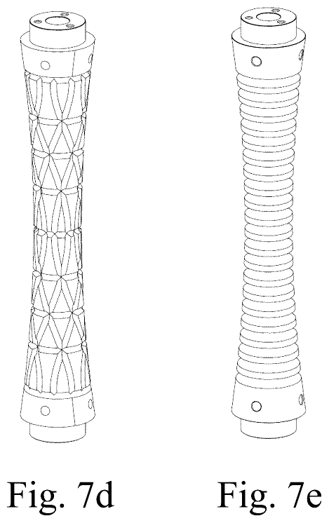

Reference is now made to FIGS. 7a to 7e presenting exemplary embodiments of articles 3a/3b (FIG. 1a). In addition to the rope-coiled embodiments (FIGS. 2a, 5a and 6), FIGS. 7a to 7e are free of a coiled rope and designed for providing a climbing experience imitating natural tree habitat conditions.

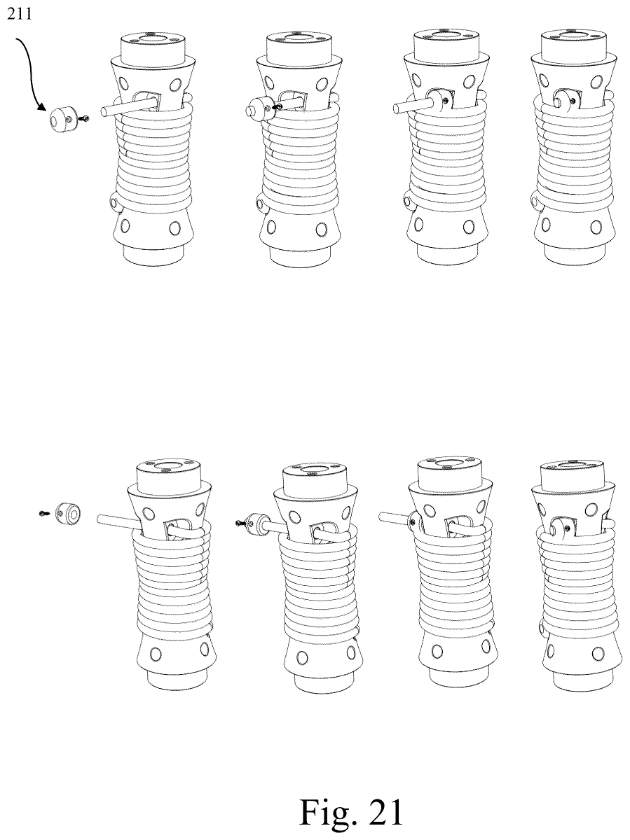

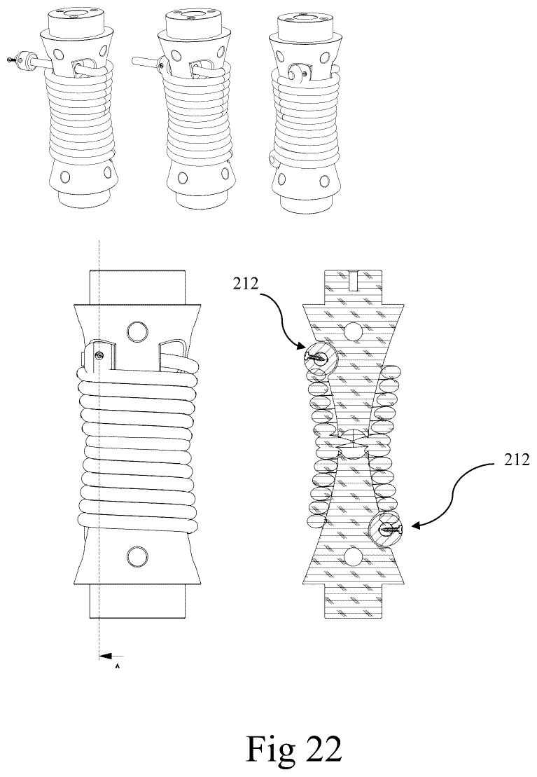

Reference is now made to FIGS. 21 and 22 presenting embodiments of a rope securing mechanism wherein a rope fastener is provided that holds the rope end with a ring 211 and a screw 212 that penetrates the rope.

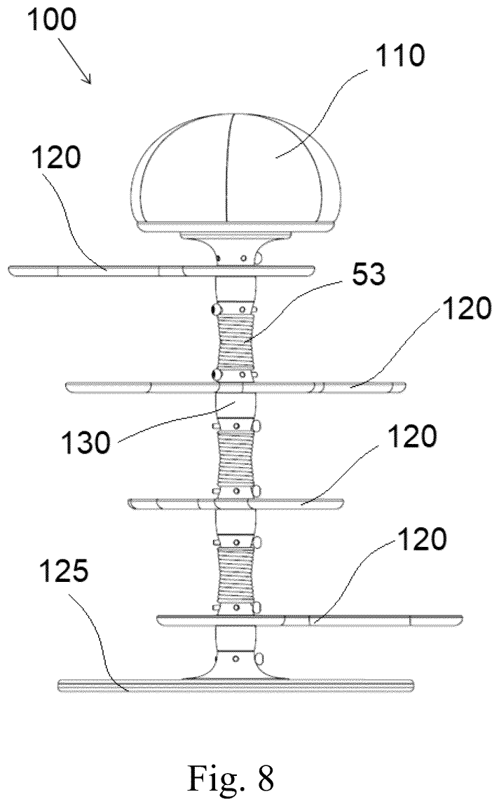

Reference now made to FIGS. 8-16 presenting alternative embodiments of an assembled piece of furniture. FIG. 8 shows embodiment 100 comprising tower 130 including at least one furniture connector of the present invention. Tower 130 is mounted on base member 125. Canopy 110 is designed for accommodating a companion animal. Numeral 120 refers to supporting members configured for supporting a companion animal, a human or an object of interest.

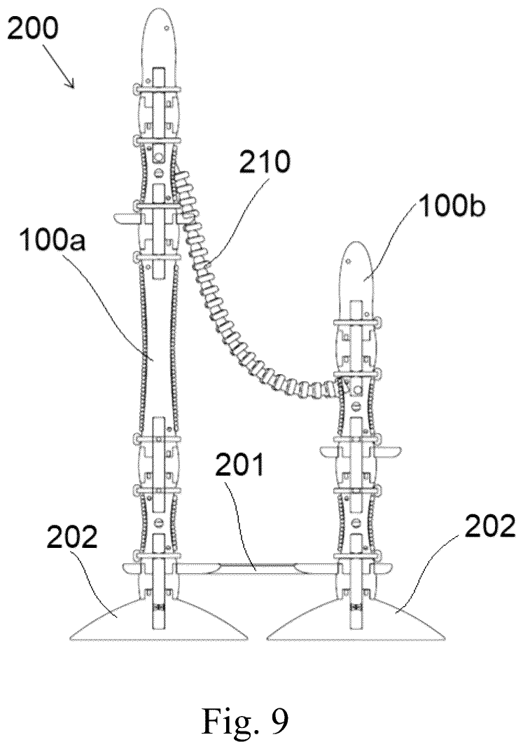

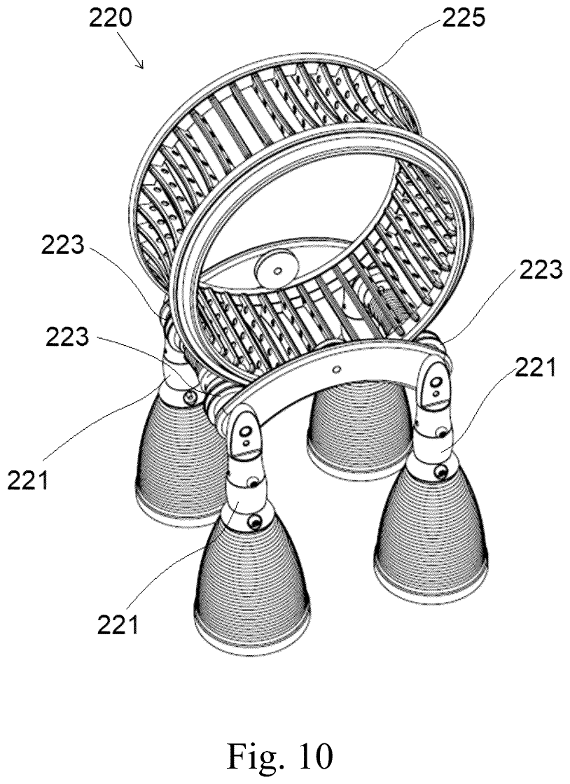

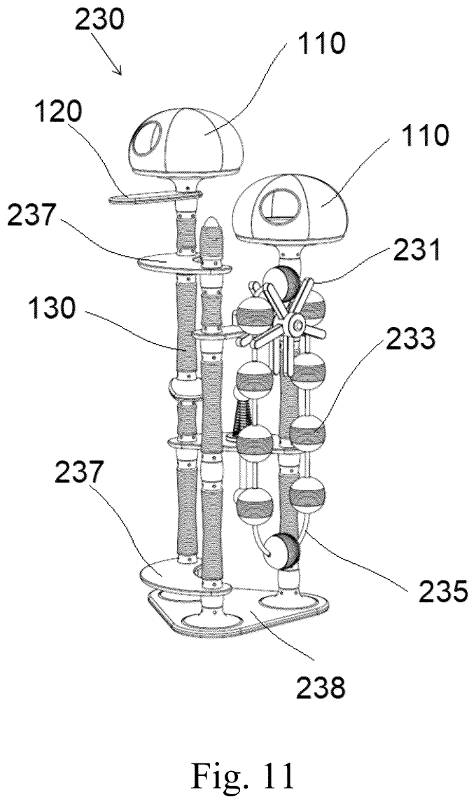

FIG. 9 presents embodiment 200 including towers 100a and 100b mounted on base members 202 and interconnected by rigid link 201. Flexible link 210 functions as a motivating and entertaining element for a companion animal. FIG. 10 shows embodiment 220 including running wheel 225 rotatable on rollers 223 supported by abutments 221. In FIG. 11, embodiment 230 includes loop wire 235 suspended on pulley 231 and provided with a plurality of entertaining elements 233 distributed along loop wire 235. Towers 130 are mounted on base member 238 and interconnected by supporting members 237.

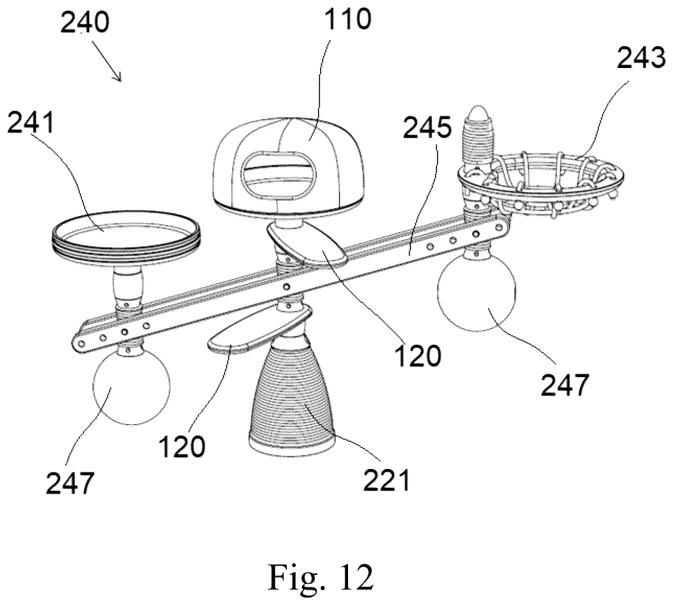

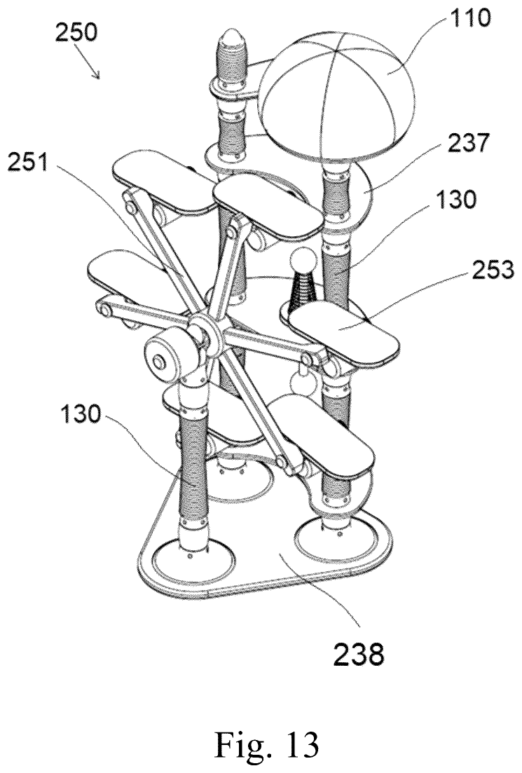

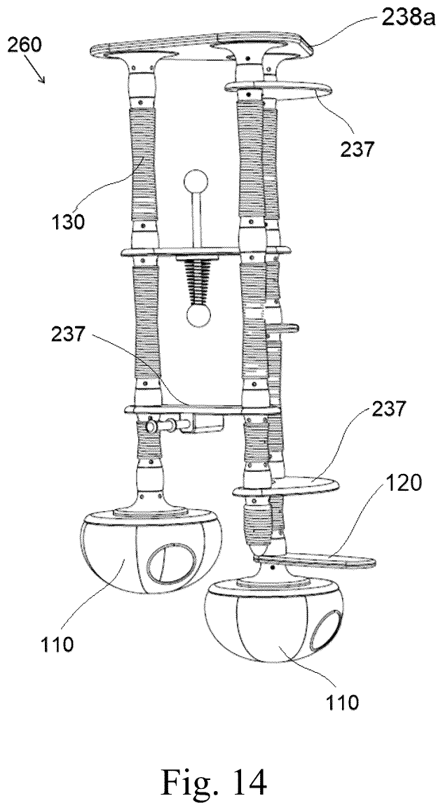

FIG. 12 shows teeter-totter embodiment 240. The aforesaid embodiment includes a supporting platform 241, basket 243 and mechanical weight bumpers 247. FIG. 13 presents observation wheel 251 having seats 253. In FIG. 14, piece of furniture 260 is secured to a ceiling (not shown).

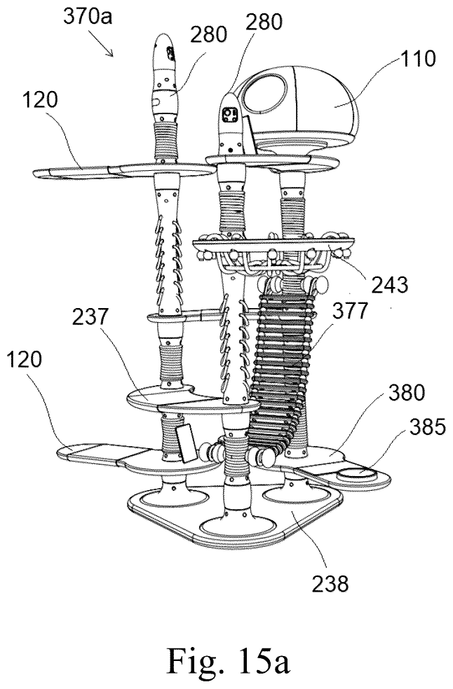

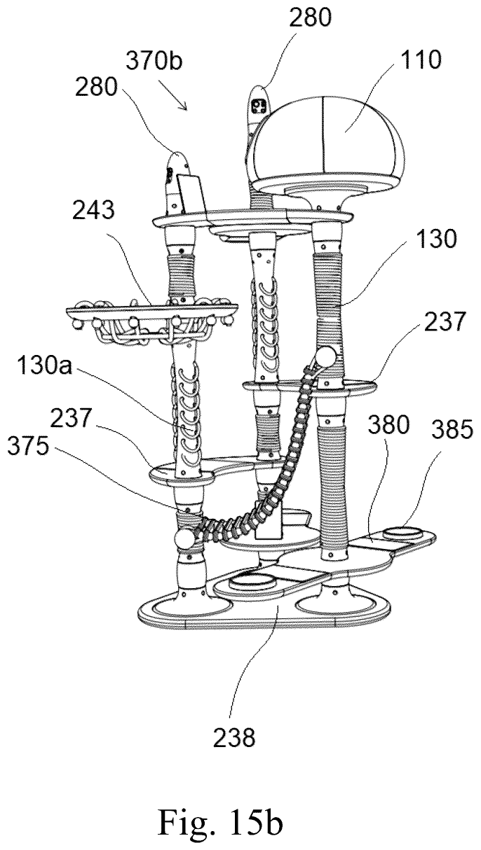

FIGS. 15a and 15b present alternative embodiments 370a and 370b provided with sensor arrangements embedded into end articles 280 described in detail below. Flexible links 375 and 377 serve for training and entertaining the habitant.

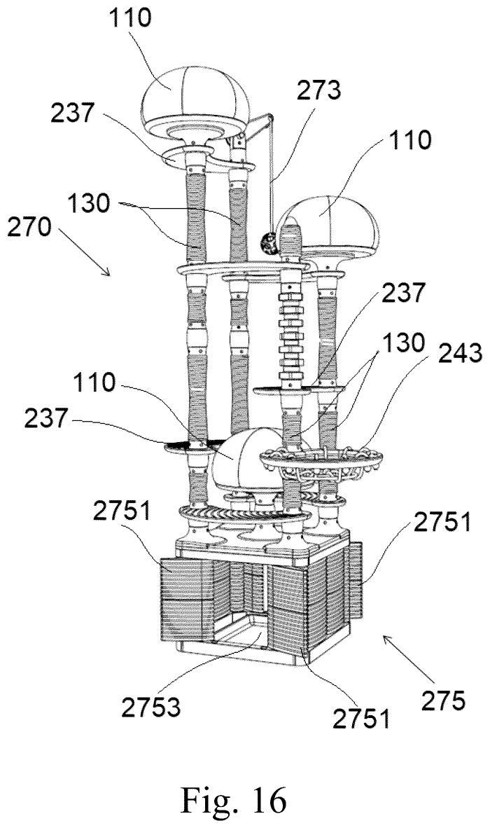

FIG. 16 presents embodiment 270 including at least one box 275 is provided with doors 2753 and configured for accommodating litter box means such as tray 2753, a self-cleaning litter box or like which is placeable into box 275.

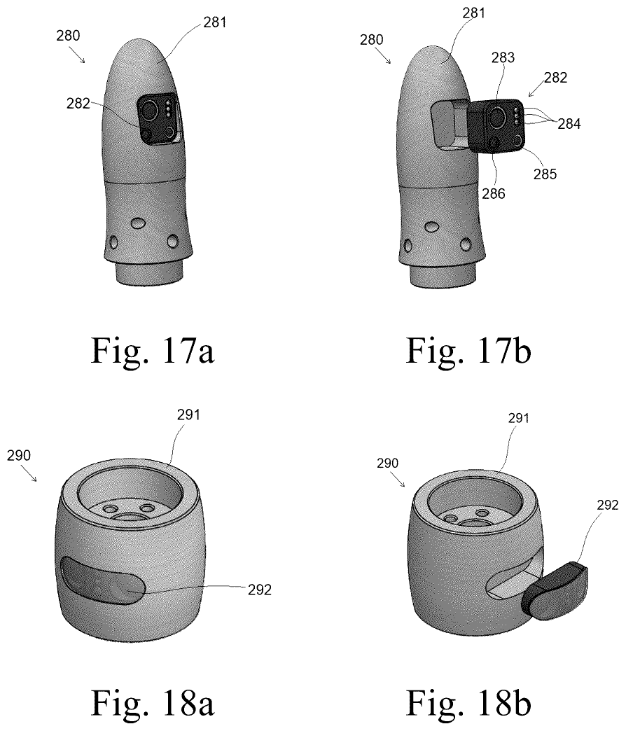

Reference is now made to FIGS. 17a and 17b presenting assembled and exploded views of end articles 280 provided with sensor arrangement 282 embedded into end article body 281. The exemplary sensor arrangement comprises camera 283, microphone 285, speaker 286 and sensors selected from a motion sensor, a temperature sensor, a blood oxygen sensor, a laser rangefinder, a CO₂ sensor, an RFID antenna, a scale, a gyroscopic sensor, a motion sensor, a pressure sensor, an illuminance sensor, a heart-beat rate sensor.

Reference is now made to FIGS. 18a and 18b presenting assembled and exploded views of intermediate member 290 provided with sensor arrangement 292 embedded into intermediate member body 281.

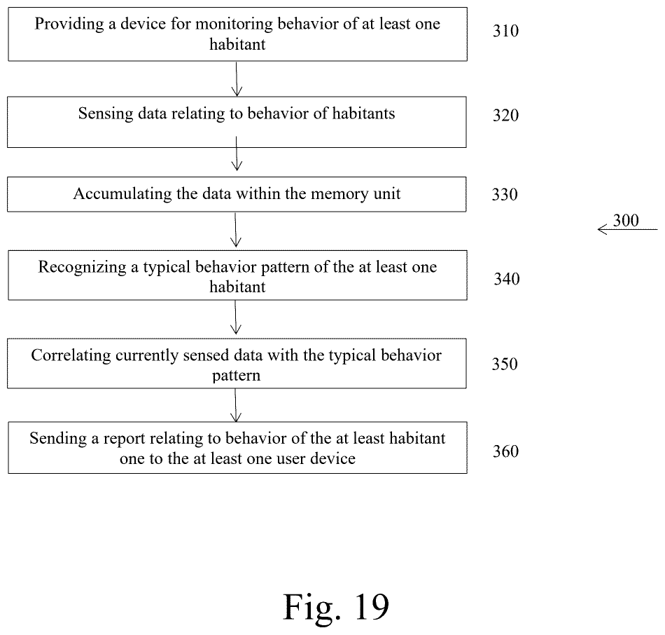

Reference is now made to FIG. 19 presenting a flowchart of computer-implemented method 300 directed to monitoring behavior of at least one habitant. The method begins at step 310 of providing a device for monitoring behavior of at least one habitant. The device includes a sensor arrangement which can include at least one of the following: a touch pad, a microphone, a camera, a motion sensor, a temperature sensor, an blood oxygen sensor, a laser rangefinder, a CO₂ sensor, an RFID antenna, a scale, a gyroscopic sensor, a pressure sensor, an illuminance sensor, a heart-beat rate sensor and any combination thereof a processor, a user unit including an interface for inputting user's instructions and outputting reports relating to monitored habitant behavior and a memory unit storing instructions to said processor. The sensor arrangement sensed data related to behavior of at least one habitant at step 320. The sensed data are accumulated in the memory (step 330). The processor algorithm recognizes a typical behavior pattern of at least one habitant in the indoor environment (step 340). The currently sensed data are correlated with the recognized typical behavior pattern (step 350). In the case of abnormality of currently sensed data, the processor sends a report relating to abnormal behavior of the at least one habitant to the at least one user device. The instructions of recognizing a typical behavior pattern of said at least one habitant and correlating currently sensed data with said typical behavior pattern comprise applying a convolutional neural network (CNN) algorithm to said accumulated data.

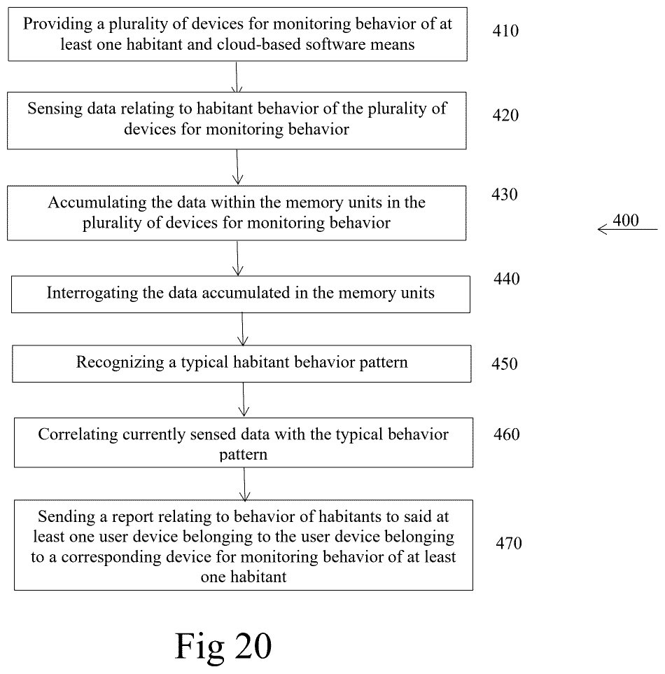

Reference is now made to FIG. 20 presenting a flowchart of computer-implemented method 400 beginning with step 410 of providing a cloud-based computer-implemented system for monitoring habitant behavior including a plurality of devices for monitoring behavior of at least one habitant as described above and cloud-based software means. Method 400 includes sensing data relating to behavior of at least one habitant by each of the monitoring devices (step 420). The sensed data are accumulated in within the memory units of monitoring devices (step 430). The accumulated data are interrogated by the cloud-based software means (step 450). The received data are analyzed in order to recognize a typical habitant behavior pattern (step 460). Currently sensed data are correlated with the recognized typical habitant behavior pattern (step 470). Abnormal data are reported to the user via a user interface belonging to the corresponding device (step 470). Method 400 includes a CNN algorithm as well.

1.-37. (canceled)

38. A furniture connector comprising:
a. first and second articles having at least one terminal portion with at least one first terminal face each; said terminal face has a blind bore thereon;
b. an intermediate member defined by contralaterally arranged first and second engaging faces; said intermediate member having a first through bore getting through said first and second engaging faces;
c. a dowel insertable into said blind bores of said first and second articles and said first through bore;
wherein said terminal face of terminal first article is engageable to first face of said intermediate member and second engaging face of said intermediate member is engageable to said terminal face of said second article by means of a plug-socket connectors such that said blind bores of said first and second articles and said first through bore of said intermediate member are coaxially arranged and configured for receiving said dowel in a releasable manner.

39. The furniture connector according to claim 38, wherein at least one of said first and second articles has a second through bore transversely oriented to said blind bore, an axisymmetrical body, or any combination thereof;
said dowel has a third through bore at at least one terminal of said dowel cooperatively disposed such that, when said dowel is inserted into said blind bore of at least one of said first and second articles, said second and third through bore are arranged coaxially and a stud releasably securable therewithin is insertable; and
said axisymmetrical body has a middle portion and terminal portions adjacent thereto; said axisymmetrical body gradually grows in diameter thereof from said middle portion to said terminal portions.

40. The furniture connector according to claim 39, wherein said third through bore has a middle bore portion and terminal portions adjacent thereto; said terminal portions are tapered such that said stud is frictionally securable in said middle portion of said third through bore.

41. The furniture connector according to claim 38, wherein at least one of said plug-socket connectors comprises a pair of surfaces facing each other; at least one of said surfaces has at least one pin projecting therefrom and releasably insertable into at least one hole on a facing surface.

42. The furniture connector according to claim 39, wherein a side surface of said axisymmetrical body is coiled with a rope in a self-compressed manner.

43. The furniture connector according to claim 42, wherein said axisymmetrical body has at least one fourth through bore having a middle bore portion and terminal portions adjacent thereto; said terminal portions are tapered; at least one terminal of said rope is inserted into said at least one forth through bore such that said rope is frictionally securable in said middle portion of said forth though bore.

44. The furniture connector according to claim 43, wherein said self compressed rope is provided with a rope fastener for holding the rope end with a ring and a screw for penetrating the rope.

45. The furniture connector according to claim 38, wherein said dowel is threadly insertable into said blind bores of said first and second articles and said first through bore.

46. The furniture connector according to claim 38, wherein at least one of said plug-socket connectors is threaded.

47. The furniture connector according to claim 38, wherein at least one of first and second articles has a second terminal face placeable on or securable to a surface of interest, said surface of interest is selected from the group consisting of a floor, a wall, a ceiling and any combination.

48. The furniture connector according to claim 38, further comprising a computer-implemented device for monitoring behavior of at least one habitant; said device comprising:
a. a sensor arrangement further comprising a sensor selected from the group consisting of a touch pad, a microphone, a camera, a motion sensor, a temperature sensor, an oxygen sensor, a heart-beat rate sensor, a laser rangefinder, a CO₂ sensor, an RFID antenna, a scale, a gyroscopic sensor, a pressure sensor, an illuminance sensor, or any combination thereof;
b. a processor;
c. at least one user unit further comprising a user interface;
d. a memory unit storing instructions to said processor to perform;
i. sensing data relating to behavior of at least one habitant;
ii. accumulating said data within said memory unit;
iii. recognizing a typical behavior pattern of said at least habitant;
iv. correlating currently sensed data with said typical behavior pattern;
v. sending a report relating to abnormal behavior of said habitant to said at least one user device;
performing said instructions of recognizing a typical behavior pattern of said habitant and correlating currently sensed data with said typical behavior pattern comprises applying a convolutional neural network algorithm to said accumulated data.

49. The furniture connector according claim to 38, wherein said wherein said sensor arrangement, processor, at least one user unit and memory unit are interconnected with each other by means of a communication protocol selected from the group consisting of: a wire connection, BlueTooth, Wi-Fi, broad band internet and any combination thereof.

50. A kit for assembling a piece of furniture or at least a part thereof; said kit comprising:
a. a plurality of furniture connectors assemblable into vertically mountable furniture assembly units; each furniture connector of said assembly unit further comprising:
i. first and second articles having at least one terminal face each; said terminal portion has a blind bore thereon;
ii. an intermediate member defined by contralaterally arranged first and second engaging faces; said intermediate member having a first through bore getting through said first and second engaging faces;
iii. a dowel insertable into said blind bores of said first and second articles and said first through bores;
said terminal face of terminal first article is engageable to first face of said intermediate member and second engaging face of said intermediate member is engageable to said terminal face of said second article by means of a plug-socket connectors such that said blind bores of said first and second articles and said first through bore of said intermediate member are coaxially arranged and configured for receiving said dowel in a releasable manner; and
b. at least one linking member configured for interconnecting said vertically mountable assembly units.

51. The kit according to claim 50, wherein at least one of said first and second articles has a second through bore transversely oriented to said blind bore, an axisymmetrical body, at least one of said plug-socket connectors comprises a pair of surfaces facing each other; at least one of said surfaces has at least one pin projecting therefrom and releasably insertable into at least one hole on a facing surface, or any combination thereof; said dowel has a third through bore at at least one terminal of said dowel cooperatively disposed such that, when said dowel is inserted into said blind bore of at least one of said first and second articles, said second and third through bore are arranged coaxially and a stud releasably securable therewithin is insertable; said axisymmetrical body has a middle portion and terminal portions adjacent thereto; said axisymmetrical body gradually grows in diameter thereof from said middle portion to said terminal portion.

52. The kit according to claim 50, wherein a side surface of said axisymmetrical body is coiled with a rope, said dowel is threadly insertable into said blind bores of said first and second articles and said first through bores, at least one of said plug-socket connectors is threaded, at least one of first and second articles has a second terminal face disposable on a surface of interest, said linking member has a supporting surface configured for accommodating a companion animal, human or objects, at least one end of said vertically mountable columns is provided with a receptacle configured for accommodating said companion animal, human or objects, and any combination thereof;
said surface of interest is selected from the group consisting of a floor, a wall, a ceiling and any combination; and said receptacle is selected from the group consisting of a canopy, a cradle, a cabin, a basket and any combination thereof.

53. The kit according to claim 50 comprising an animating element selected from the group consisting of a flexible linking element interconnecting at least two of said furniture assembly units, a running wheel, a suspended object, an observation wheel, a teeter-totter, a loop wire suspended on a pulley and provided with a plurality of entertaining elements distributed along said loop wire, ladder and any combination.

54. The kit according to claim 50 comprising a base box accommodating a litter tray.

55. The kit according to claim 50, further comprising a computer-implemented device for monitoring behavior of at least one habitant; said device comprising:
a. a sensor arrangement further comprising a sensor selected from the group consisting of a touch pad, a microphone, a camera, a motion sensor, a temperature sensor, an oxygen sensor, a heart-beat rate sensor, a laser rangefinder, a CO₂ sensor, an RFID antenna, a scale, a gyroscopic sensor, a pressure sensor, an illuminance sensor, or any combination thereof;
b. a processor;
c. at least one user unit further comprising a user interface;
d. a memory unit storing instructions to said processor to perform:
i. sensing data relating to behavior of at least one habitant;
ii. accumulating said data within said memory unit;
iii. recognizing a typical behavior pattern of said at least habitant;
iv. correlating currently sensed data with said typical behavior pattern;
v. sending a report relating to abnormal behavior of said habitant to said at least one user device;
performing said instructions of recognizing a typical behavior pattern of said habitant and correlating currently sensed data with said typical behavior pattern comprises applying a convolutional neural network algorithm to said accumulated data.

56. The kit of claim 50, wherein said wherein said sensor arrangement, processor, at least one user unit and memory unit are interconnected with each other by means of a communication protocol selected from the group consisting of: a wire connection, BlueTooth, Wi-Fi, broad band internet and any combination thereof.
